(19) United States
(12) Patent Application Publication
(10) Pub. No.: US 2018/0020904 A1
(43) Pub. Date: Jan. 25, 2018

(54) IMAGING UNIT FOR AN ENDOSCOPE, AND METHOD FOR PRODUCING AN IMAGING UNIT

(71) Applicant: OLYMPUS WINTER & IBE GMBH, Hamburg (DE)

(72) Inventors: Martin WIETERS, Hamburg (DE); Uwe SCHOELER, Hoisdorf (DE)

(73) Assignee: OLYMPUS WINTER & IBE GMBH, Hamburg (DE)

(21) Appl. No.: 15/708,876

(22) Filed: Sep. 19, 2017

Related U.S. Application Data
(63) Continuation of application No. PCT/EP2016/054626, filed on Mar. 4, 2016.

(30) Foreign Application Priority Data
Mar. 25, 2015 (DE) 10 2015 205 457.8

(51) Int. Cl. A61B 1/05; G02B 23/24; A61B 1/00

(52) U.S. Cl. CPC A61B 1/051; A61B 1/00096; G02B 23/243; A61B 1/0011; H04N 2005/2255

(57) ABSTRACT

An imaging unit including: a guide tube having an inner lateral surface extending in a longitudinal direction of the guide tube, the inner lateral surface defining an inner chamber; a lens tube having an outer lateral surface extending in a longitudinal direction of the lens tube, the lens tube being at least sectionally accommodated within the inner chamber of the guide tube; and at least one optical element which is accommodated in the lens tube; wherein the lens tube comprises a plurality of bars disposed on the outer lateral surface, each of the plurality of bars interacting with a corresponding one of a plurality of grooves recessed in the inner lateral surface of the guide tube.

### CROSS-REFERENCE TO RELATED APPLICATION

The present application is a continuation of PCT/EP2016/054626 filed on Mar. 4, 2016, which is based upon and claims the benefit to DE 10 2015 205 457.8 filed on Mar. 25, 2015, the entire contents of each of which are incorporated herein by reference.

### BACKGROUND

### Field

The present application relates to an imaging unit comprising at least one optical element that is accommodated in a lens tube. The present application further relates to an endoscope as well as a method for producing an imaging unit.

### Prior Art

In optical imaging systems, such as video endoscopes, the lens is focused by transverse displacement, i.e., by a displacement along its optical axis, relative to a plane in which a sharp image is desired and in which, e.g., an image sensor is located. Then the lens such as the endoscope lens is permanently fixed by, e.g., being glued. In practice, primarily rotationally symmetrical cylindrical fits are used to align the optical components. Despite minimal tolerances in the fit, it is possible for the optical element or the optical unit to tilt slightly. Increasingly stringent demands are always being posed on the coaxiality between a normal of the image sensor and the optical axis of the imaging optical element which, for example, is part of an endoscope lens, especially in high-resolution optical units, so that the achievable image quality can be fully exploited.

To satisfy these high demands, it would be possible to increase a guide length in the fit. However, this simultaneously leads to a loss of light and possibly greater disper-sion. Further reducing the fit tolerances only theoretically allows a potential tilt to be reduced since there must be a minimum play in the fit to install the optical element.

### SUMMARY

It is an object to present an imaging unit, an endoscope, and a method for producing an imaging unit, wherein the imaging unit comprises an optical element, and wherein a precise alignment of the optical element is possible.

Such object can be solved by an imaging unit, for example, for an endoscope, comprising at least one optical element which is accommodated in a lens tube, and wherein the lens tube is at least sectionally accommodated in an inner chamber surrounded by a guide tube, wherein the lens tube comprises a plurality of bars on an outer lateral surface that extend in a longitudinal direction of the lens tube and each interacts with a groove recessed in an inner lateral surface of the guide tube.

In the disclosed embodiments, a cylindrical fit between the lens tube and guide tube is abandoned and the fit is not designed entirely rotationally symmetrical. Along the perimeter of the lens tube, bars are provided on an outer lateral surface of the lens tube. These bars at least sectionally expand the lens tube parallel to its longitudinal direction. The second part of the fit, i.e., the guide tube, is provided with corresponding grooves to accommodate the bars. In the peripheral direction, a groove is designed wider than the bars. A simple and io non-clamping installation of the lens tube in the guide tube is accordingly possible, i.e., by inserting the lens tube along the longitudinal direction into the guide tube. The optical element can be an imaging optical component of an endoscope lens. It is also possible for an endoscope lens to be provided as the optical element. The optical element, or more precisely the lens tube in which it is accommodated, is displaced in the longitudinal direction until the desired position is reached in which it images sharply, for example, on the image sensor.

According to an embodiment, the bars and the grooves have complementary shapes in a cross-section lying perpendicular to the longitudinal direction. Bars and grooves configured with shapes that are complementary with each other allow a precise self-centering of the lens tube in the guide tube. The bars can be arranged evenly distributed along the perimeter of the lens tube. For example, three bars can be provided at a spacing of 120° along the perimeter of the lens tube on its outer lateral surface. The same holds true for the associated grooves in the guide tube. According to other embodiments, different numbers of bars and grooves can be provided, wherein their number is of course always identical. For example, two, four, five or more bars, or respectively grooves, are on/in the lens tube, or respectively the guide tube.

According to another embodiment, the bars and/or the grooves can be trapezoidal in a cross-section lying perpendicular to the longitudinal direction, wherein at least one side flank of the bar and/or at least one side wall of the groove is angled toward a center of the bar or respectively the groove, so that in the cross-section, the bar tapers toward its end face and/or the groove narrows toward its base.

By rotating the lens tube and the guide tube relative to each other, the bars and the grooves, or more precisely the side flanks of the bars and the side walls of the grooves, come into contact with each other. Since the side flanks of the bars and the side walls of the grooves are angled, a specific pressure between the two surfaces is realized by exerting a pre-determined torque, wherein they slide slightly on each other. The two parts are accordingly centered relative to each other. As a result, the lens tube is aligned tilt-free and securely within the guide tube. Then the lens tube is displaced within the guide tube so that the optical element in the lens tube is imaged sharply, for example, on an image sensor. The torque which is used for centering can be selected to be large enough so that a clamping seat at least temporarily exists between the two components at the same time as the self-centering of the lens tube in the guide tube.

The chosen inclination at which the side flank of the bar and the side wall of the groove is angled is such that there is a sufficiently large pressure between the lens tube and the guide tube in a special application so that the desired self-centering occurs. The inclinations at which the side flank of the bar and the side wall of the groove are angled can be at least approximately equivalent.

Moreover, the chosen torque is only large enough for the friction between the lens tube and the guide tube to be sufficient to prevent the optical element from tilting relative to the image sensor; however, the lens tube can still be slightly displaceable in the longitudinal direction relative to the guide tube. It is accordingly possible to adjust the focus position. Only afterward is the lens tube fixed on or in the guide tube.

The image sensor, which can comprise the imaging unit, can be aligned perpendicular to a longitudinal direction of the guide tube. In addition, the guide tube and the image sensor can be arranged in a fixed spatial relationship relative to each other.

The imaging unit can be provided both for endoscopes with a rigid shaft as well as for endoscopes with a flexible shaft. In addition, the imaging unit can be used in an endoscope. However, the use of the imaging unit is not restricted to endoscopes. It can also be used in cameras, camera modules, lighting and imaging systems.

The lens tube can be configured integrally, or respectively monobloc together with its bars, moreover, in either the integral or monobloc configuration, the lens tube can be formed of the same material. The longitudinal direction of the guide tube as well as the longitudinal direction of the lens tube can correspond to a respective direction of longitudinal extension of the component. In the optimally centered state of the lens tube, its longitudinal direction can correspond with the longitudinal direction of the guide tube, at least approximately. Thus, the two longitudinal directions extend at least approximately in a common direction, in other words, can be minimally offset parallel to each other.

The bars can extend at least sectionally along the longitudinal direction of the lens tube. For example, the bar can be designed to run in an interrupted manner along the length of the lens tube. The bars can extend along the entire length of the lens tube in its longitudinal direction. The same holds true for the grooves that can also extend sectionally, such as, along the entire length of the guide tube in its longitudinal direction.

According to an additional embodiment, the imaging unit can be configured in that the at least one side flank of the bar and/or the at least one side wall of the groove are angled at a predetermined inclination relative to a radial direction, wherein the radial direction is assumed to be a direction which runs radially starting from a center of the lens tube, or respectively the guide tube, and penetrates the middle of the end face of the bar, or respectively the base of the groove in the cross-section.

Both side walls of the groove and/or both side flanks of the bar can be angled by the predetermined, i.e., at least approximately identical, inclination relative to the radial direction. In other words, the bar and the groove can be designed symmetrical. It is possible to optionally achieve a centering of the lens tube in the guide tube by a clockwise or counterclockwise rotation.

The predetermined inclination can lie between 30° and 60°, such as, between 35° and 55°, between 40° and 50°, or at least approximately 45°. When the inclinations are too small, the self-centering forces are too slight; whereas when the inclinations are too large, excessive static friction can arise between the lens tube and the guide tube. The smaller of the two inclinations is always understood to be the inclination between the angled side flank of the bar, or respectively the angled side wall of the groove, and the radial direction, viewed from an intersection between the plane of the side flank, or respectively the side wall and the radial direction. In this context, this disclosure references the radial direction or a direction parallel thereto. The radial direction extends from a center of the guide tube, or respectively the lens tube, and penetrates the center, or respectively the middle of the base of the groove, or respectively the end face of the bar. If the guide tube and the lens tube are ideally centered relative to each other, the radial direction of the guide tube and the radial direction of the lens tube coincide. If the centering is not ideal, reference is made to the radial direction of the lens tube to determine the inclination of the flank, and reference is made to the radial direction of the guide tube to determine the inclination of the side wall.

The lens tube and the guide tube can be formed from metal and/or plastic. The same or different materials can be provided for the lens tube and the guide tube.

Where the lens tube and the guide tube are each made of steel or another metal material, they can be welded, soldered or glued to each other after centering and adjusting the optical element. Welding or soldering can be carried out, for example, with the assistance of a laser. For this purpose, welding or soldering points, for example, can be provided in a fillet between an outer lateral surface of the lens tube and an end face of the guide tube. These connecting points can be arranged evenly distributed along the perimeter. For example, three connecting points that are each spaced from each other at an angle of 120° can be along the perimeter of the lens tube.

The lens tube and guide tube can be adhered by introducing a low-viscosity adhesive into a gap between the lens tube and the guide tube. Such an adhesive can be hardened under the effect of UV radiation. For this purpose, the outer guide tube can be configured as translucent, or formed of a translucent material. A material can be used that is translucent to the UV radiation. By irradiating the guide tube from its exterior, the UV radiation is coupled into the adhesive to harden it.

According to another embodiment, the guide tube can be formed of a translucent material, and the lens tube can be formed of a material that is strongly light-absorbent. Depending on the wavelength of the light used, such as laser light, a material that is translucent to this wavelength, or a strongly absorbent material can be chosen. The lens tube and the guide tube can be welded to each other as the laser light penetrates the guide tube and is strongly absorbed by the material of the lens tube so that it melts locally.

Moreover according to another embodiment, the guide tube can comprise an outer groove which extends in the longitudinal direction of the guide tube and is recessed in an outer lateral surface of the guide tube, wherein, the outer groove can extend in a region of the guide tube so that it at least partially overlaps the groove recessed in the inner lateral surface in the peripheral direction; moreover, i the outer groove in the peripheral direction can at least have a width of the groove recessed in the inner lateral surface.

Where the lens tube and the guide tube are welded to each other, the lens tube and the guide tube can be made of thermoplastic materials for this purpose that furthermore have in particular similar thermal properties. The guide tube can be translucent to the laser beam which is used such as UV or IR radiation, or to radiation in the visible range. The lens tube can be highly absorbent to the corresponding wavelength, for example, made of a material with a black color such as plastic. The outer groove can reduce the thickness of the material of the guide tube in the relevant region so that unnecessarily high absorption of the laser beam does not occur therein. The beam can be, therefore, coupled in. The laser beam that penetrates the guide tube in the region of the groove heats the lens tube underneath, a section of its material is melted, and consequently, the two components are welded together in this region.

Likewise, it is possible to harden a UV cross-linking adhesive in the gap between the lens tube and the guide tube with the assistance of a (UV) laser beam coupled into the region of this gap through the guide tube. The same holds true for other wavelengths.

Such object can be further solved by an endoscope comprising an imaging unit according to one or more of the previously cited embodiments. The endoscope can have a rigid or flexible shaft. The same or similar advantages apply to the endoscope as with the imaging unit.

Moreover, such object can be solved by a method for producing an imaging unit according to one or more of the aforementioned embodiments, wherein the method comprises:
at least sectional introduction of the lens tube into the inner chamber surrounded by the guide tube, wherein a bar engages in a groove,
the optical element is fixed by connecting the lens tube to the guide tube.

Such method can allow the optical element to be quickly and precisely adjusted and centered.

The same or similar advantages and features with respect to the imaging unit also apply to the method.

The bars and/or the grooves can be trapezoidal in a cross-section lying perpendicular to the longitudinal direction, wherein at least one side flank of the bar and/or at least one side wall of the groove is angled toward a center of the bar or respectively the groove, so that in the cross-section, the bar tapers toward its end face and/or the groove narrows toward its base, wherein the method further comprises to adjust the optical element accommodated in the lens tube; the lens tube is rotated about its longitudinal direction relative to the guide tube so that the angled side flank of the bar comes into contact with the angled side wall of the groove, and the optical element is centered; the lens tube is displaced relative to the guide tube in the longitudinal direction of the guide tube to set a desired longitudinal position of the lens tube.

The lens tube and guide tube can be welded, soldered and/or glued to each other under the effect of laser radiation, wherein the guide tube is made of a material translucent to the laser radiation, and the lens tube is made of a material that strongly absorbs the laser radiation, and wherein the guide tube is irradiated with the laser radiation in the region of the outer groove to weld, solder and/or glue the lens tube and the guide tube.

According to another embodiment, an imaging unit, such as an imaging unit of an endoscope, can be provided that is made using a method according to one or more of the aforementioned features.

### BRIEF DESCRIPTION OF THE DRAWINGS

Further characteristics will become apparent from the description of embodiments together with the claims and the included drawings. Embodiments can fulfill individual characteristics or a combination of several characteristics.

The embodiments will be described below, without restricting the general idea of the invention, based on exemplary embodiments in reference to the drawings, wherein we expressly refer to the drawings with regard to the disclosure of all details that are not explained in greater detail in the text. In the following:

FIG. 1 illustrates an endoscope in a schematically simplified side view,

FIG. 2 illustrates an imaging unit in a schematically simplified longitudinal view,

FIG. 3 illustrates a schematically simplified cross-sectional view along line III-III in FIG. 2,

FIGS. 4 and 5 illustrate schematically simplified detailed views of the cross-section of FIG. 3 that illustrate additional exemplary embodiments.

In the drawings, the same or similar types of elements and/or parts are provided with the same reference numbers so that a re-introduction is omitted.

### DETAILED DESCRIPTION

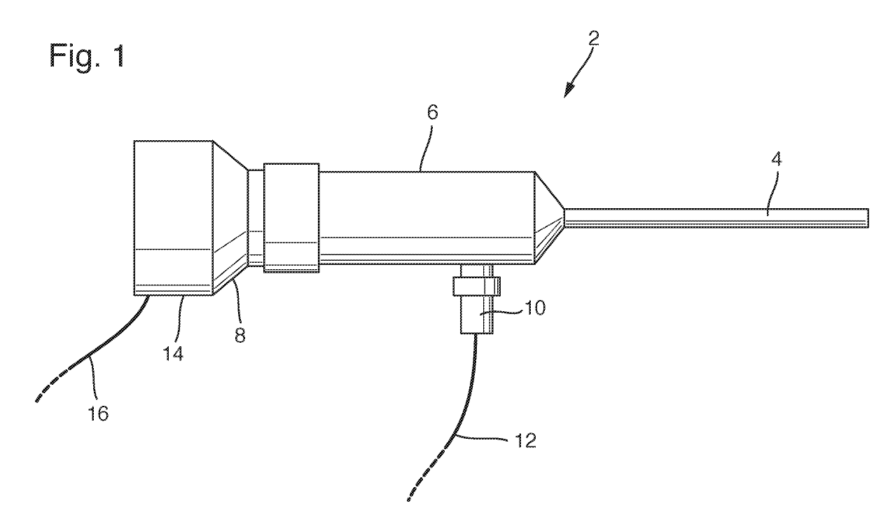

FIG. 1 illustrates a schematic and simplified side view of an endoscope 2, such as, a video endoscope. On its distal end, the endoscope 2 comprises a tubular shaft 4 in which an optical element, such as, an endoscope lens is arranged. With the assistance of the endoscope lens, a surgical and investigative region is observed, or respectively depicted, which lies distally in front of a free end of the shaft 4. Starting from the endoscope lens and moving proximally, the image is passed on by relay lenses through the shaft 4 which terminates in a housing 6. With endoscopes 2 that have a flexible shaft 4, a flexible bundle of optical fibers is provided as the relay lens system.

At the proximal end of the endoscope 2 is a housing 6 with an eyepiece 8. The housing 6 serves for handling the endoscope 2. On the side of the housing 6 is a light source 10, such as, an LED light source. The LED light source is connected by a connecting cable 12 to a suitable power supply.

A schematically portrayed camera head 14 with an ocular adapter (not shown) is arranged on the eyepiece 8. The camera head 14 detects the light exiting the endoscope 2 with an image sensor. The camera head 14 is supplied with power by means of a connection 16. Furthermore, it is possible to send image signals by the connection 16 from the surface sensor of the camera head 14 to an external evaluation unit and transmit control signals to the camera head 14.

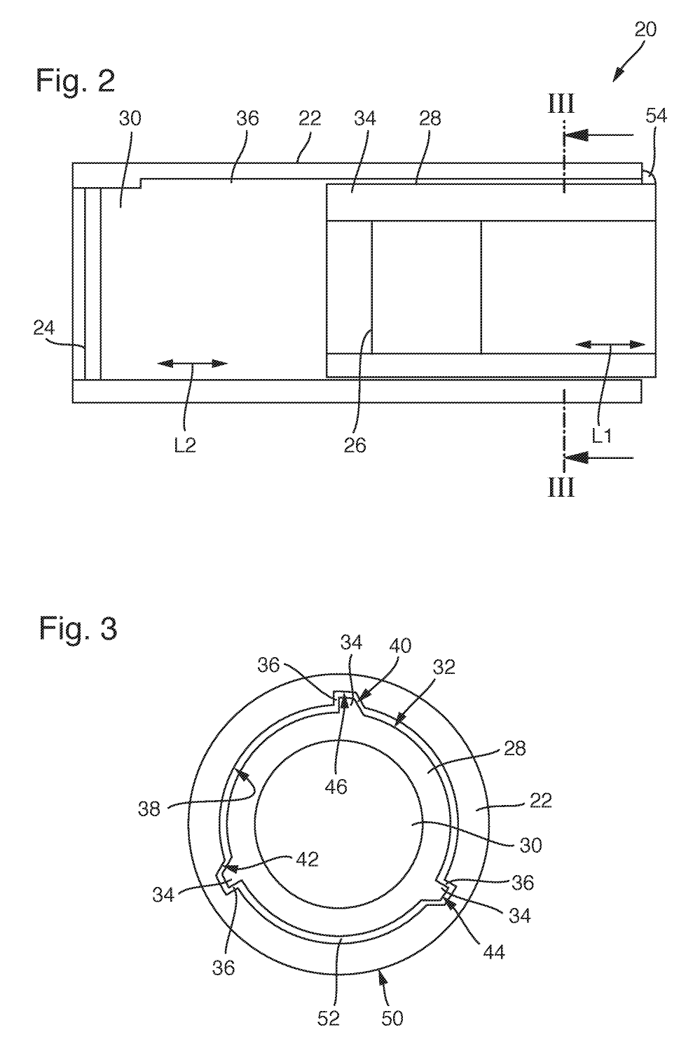

The endoscope 2 has an imaging unit which comprises an optional image sensor 24 and an optical element 26. FIG. 2 illustrates the imaging unit 20 in a schematically simplified longitudinal view. For the sake of clarity, relay lenses that may exist are not represented. The imaging unit 20 comprises a guide tube 22 which is in a fixed spatial relationship to the image sensor 24, such as a flat image sensor, which can be a CCD or CMOS sensor. The image sensor 24 is arranged in the guide tube 22 only as an example in the depicted exemplary embodiment. Moreover, the imaging unit 20 comprises at least one optical element 26 such as a lens, lens group or an endoscope lens. The optical element 26 is accommodated in a lens tube 28. Moreover, the lens tube 28 is, or respectively can be accommodated at least sectionally in an inner chamber 30 enclosed by the guide tube 22.

FIG. 3 illustrates a schematically simplified cross-sectional view along the line identified as III-III in FIG. 2. On its outer lateral surface 32, the lens tube 28 comprises a plurality of bars 34. The bars 34 each extend in a longitudinal direction L1 of the lens tube 28 and interact with corresponding grooves 36 which are recessed in an interior lateral surface 38 of the guide tube 22. The grooves 36 extend in a longitudinal direction L2 of the guide tube 22. In the centered state illustrated in FIGS. 2 and 3, the longitudinal direction L1 of the lens tube 28 and the longitudinal direction L2 of the guide tube 22 coincide. Although FIG. 3 illustrates the bars 34 being on the outer lateral surface 32 of the lens tube 28 and the grooves 36 being recessed in the interior lateral surface 38 of the guide tube 22, this disclosure further contemplates a reverse configuration where the bars 34 are on the inner lateral surface 38 of the guide tube 22 and the grooves 36 are recessed in the outer lateral surface 32 of the lens tube 28.

The bars 34 and the grooves 36 are trapezoidal in the cross-section depicted in FIG. 3 which is oriented perpendicular to the longitudinal directions L1, L2. A side flank 40 of the bars 34 in a side wall 42 of the groove 36 is angled toward a center of the respective bar 34, or respectively the respective groove 36 so that in the depicted cross-section, the bar 34 tapers toward its end face, and the groove 36 narrows towards its base 46. For reasons of clarity, only one side flank 40 and one side wall 42 are provided with reference numbers.

The lens tube 28 and the bars 34 on its outer lateral surface 32 can be configured integrally, or respectively monobloc, of the same material with the lens tube 28, or respectively with the guide tube 22. A plastic or metal can be provided as the material for the lens tube 28. The same holds true for the guide tube 22 that can also be made of a plastic or metal.

One of the grooves 36 recessed in the guide tube 22 in which the associated bar 34 of the lens tube 28 extends is also visible in the longitudinal section depicted in FIG. 2. The groove 36 can extend sectionally along the longitudinal direction L2 of the guide tube 22. It is also provided that the groove 36 can extend along the entire length of the guide tube 22 in its longitudinal direction L2. The bar 34 of the lens tube 28 can extend along the entire length of the lens tube 28 in its longitudinal direction L1. According to another embodiment (not shown), the bar 34 can only extend sectionally in the longitudinal direction L1 of the lens tube 28.

In the exemplary embodiment depicted in FIG. 3, the bars 34 and grooves 36 are arranged evenly distributed along the perimeter of the lens tube 28, or respectively the guide tube 22. For example, they have a spacing of 120° along the respective perimeter of the lens tube 28, or respectively the guide tube 22. According to other exemplary embodiments (not shown), different numbers of bars 34, or respectively grooves 36 can be provided such as two, four, five or more, which can also be distributed evenly along the perimeter of the lens tube 28, respectively the guide tube 22.

The bars 34 of the objective tube 28 and the grooves 36 of the guide tube 22 can be configured to have a complementary shape in the cross-section depicted in FIG. 3. For example, the bars 34 as well as the grooves 36 can have the shape of a rectangular trapezoid. Consequently, in such configuration only one side flank 40 of the bars 34 and only one side wall 42 of the grooves 36 are angled. The lens tube 28 is centered in the guide tube 22 by rotating the lens tube 28 clockwise relative to the guide tube 22. As a result of this rotation, the side flank 40 of a bar 34 comes into contact with the side wall 42 of the groove 36. If a predetermined torque is exerted on the lens tube 28 or the guide tube 22 during this rotation, a centering force arises that is directed toward the center of the lens tube 28 as a consequence of the surfaces sliding on each other.

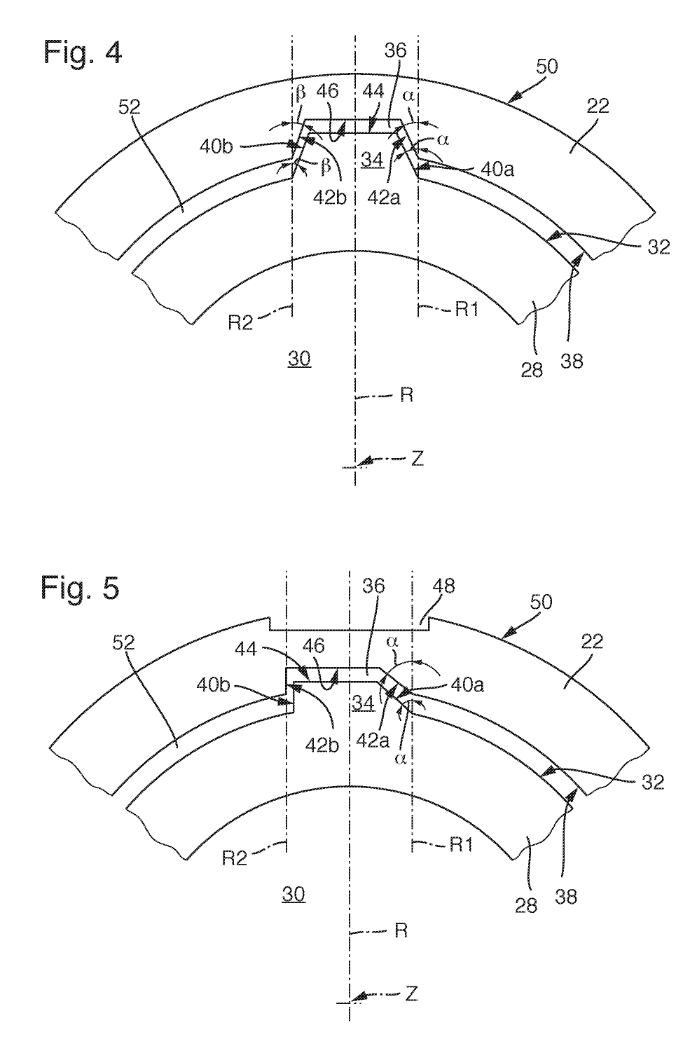

FIG. 4 illustrates a schematically simplified detailed view of the guide tube 22 and the lens tube 28 in the region of the bar 34, or respectively the groove 36. In the depicted exemplary embodiment, the bar 34 and the groove 36 have two angled side flanks 40a, 40b, or respectively two angled side walls 42a, 42b. It is accordingly possible to center the lens tube 28 relative to the guide tube 22 both by a clockwise rotation as well as a counterclockwise rotation. For example, a first side wall 42a of the groove 36 and a first side flank 40a of the bar 34 is angled at a first inclination α, and a second side wall 42b of the groove 36, and a second side flank 40b of the bar 34 is angled at a second inclination α, β relative to a radial direction R. The first and second inclination α, β can be the same or a different angle.

The radial direction R is a direction that runs radially from a center Z of the lens tube 28, or respectively the guide tube 22, and penetrates the end face 44 of the bar 34, or respectively the base 46 of the groove 36 in the depicted cross-section. The respective inclinations α, β at which the side flanks 40a, 40b, or respectively the side walls 42a, 42b of the bar 34, or respectively the groove 36 are angled are measured relative to this direction. For this purpose, a first parallel direction R1 and a second parallel direction R2 are drawn in FIG. 4 in a dot-dashed line. The two parallel directions R1, R2 are directions that are displaced parallel to the radial direction R. The inclination of the side flanks 40a, 40b is measured relative to these parallel directions R1, R2.

An inclination of the flanks 40a, 40b of the bar 34 is measured relative to the parallel directions R1, R2 that runs from the center Z of the lens tube 28 through a foot of the bar 34 at the transition between the outer lateral surface 50 and the respective flank 40a, 40b. An inclination of the side walls 42a, 42b of the groove 36 is measured relative to the parallel directions R1, R2 that runs from the center Z of the lens tube 28 through a top edge of the groove 36 at the transition between the inner lateral surface 38 and the respective side wall 42a, 42b. In the depicted exemplary embodiment in which the lens tube 28 and guide tube 22 are ideally centered, the corresponding points coincide for example so that only two parallel directions R1, R2 are needed to determine the angle of inclination α, β. For example, the lens tube 28 is arranged concentric to the guide tube 22 so that they have a common center Z.

The angles of inclination α, β of the side flanks 40a, 40b and the side walls 42a, 42b can be at least approximately identical. The angle of inclination α, β can be between 30° and 60°, between 35° and 55°, between 40° and 50°, and at least approximately 45°. The aforementioned angular ranges have proven to be advantageous since the centering forces acting on the lens tube 28 are too small when the angles are too large, whereas they are too large when the angles are too small.

FIG. 5 illustrates another schematic and simplified detailed view of the lens tube 28 and the guide tube 22 in the region of the bar 34, or respectively the groove 36 according to another exemplary embodiment. The bar 34 and the groove 36 in the illustrated cross-section have the shape of a rectangular trapezoid so that the lens tube 28 can be centered relative to the guide tube 22 by a clockwise rotation. Correspondingly, a first side flank 40a and a first side wall 42a of the bar 34 or respectively the groove 36 is angled at an inclination a that is for example 45°. The second side wall 40b of the bar 34 and the second side wall 42b of the groove 36 are contrastingly oriented vertically, i.e., they extend in the direction of the radial direction R, or stated more precisely, along the second parallel direction R2 that is displaced parallel relative to the radial direction R.

The guide tube 22 comprises an outer groove 48 that extends in the longitudinal direction L2 of the guide tube 22 and is recessed in an outer lateral surface 50 of the guide tube 22. The outer groove 48 is configured so that it extends in such a region of the guide tube 22 so that, in a peripheral direction, it at least partially overlaps the groove 36 recessed in the inner lateral surface 38. In the exemplary embodiment, the outer groove 48 entirely overlaps the groove 36, and accordingly has at least the width of the groove 36 that is recessed in the inner lateral surface 38. For example, the width of the outer groove 48 measured in a peripheral direction is larger than the maximum width of the groove 36 measured in a peripheral direction.

Moreover, the guide tube 22 can be made of a translucent material, and the lens tube 28 can be made of a material that is very light-absorbent. For example, a translucent plastic can be used to produce the guide tube 22, whereas the lens tube 28 is made of a blackened plastic. This makes it possible to weld the lens tube 28 and the guide tube 22 together, for example with the assistance of laser radiation after the lens tube 28 has been centered. The highly absorbent material of the lens tube absorbs the laser radiation and is accordingly regionally melted. After subsequent cooling, the lens tube 28 is fixed to the guide tube 22. Advantageously, the laser radiation is not, or is only slightly, absorbed by the material of the guide tube 22 since it is made of a translucent material. A material is selected that is largely transparent to the wavelength used, such as UV light. The lens tube 28 and the guide tube 22 can moreover be soldered or respectively glued to each other.

The lens tube 28 and the guide tube 22 are produced in such a manner that there is a gap 52 between them. The lens tube 28 can be easily introduced into the guide tube 22. The fit between the two components, especially the width of the gap 52, is selected to facilitate easy assembly. In such configuration, it is unnecessary to design a particularly tight fit between the components since it does not directly influence the subsequent centering of the lens tube 28.

According to a method for producing an imaging unit 20 as described in the aforementioned exemplary embodiments, first the lens tube 28 is at least sectionally introduced into the inner chamber 30 surrounded by the guide tube 22, wherein respectively one bar 34 of the objective lens tube 28 engages in a groove 36 of the guide tube 22. Then the optical element 26 such as an endoscope lens or an optical component thereof accommodated in the lens tube 28 is adjusted relative to the image sensor 24.

This is accomplished in a first step by rotating the lens tube 28 about its longitudinal direction L1 relative to the guide tube 22 so that the at least one angled side flank 40 of the bar 34 comes into contact with the at least one angled side wall 42 of the groove 36. By applying a predetermined torque, the optical element 26 is centered relative to the image sensor 24. In this context, there is also enough static friction between the angled side flank 40 of the bar 34 and the angled side wall 42 of the groove 36 so that the lens tube 28 is at least temporarily held in the guide tube 22. This measure is however optional.

Then in a subsequent second step, in particular the lens tube 28 including the optical element is advanced in the longitudinal direction L2 of the guide tube 22. In the desired and set end positions, the optical element 26 can be sharply imaged on the image sensor 24. In other words, it is at least approximately within the image plane of the optical element 26.

Then the optical element 26 is fixed relative to the image sensor 24 by connecting the lens tube 28 to the guide tube 22.

The lens tube 28 is fixed in the guide tube 22 for example by welding or soldering the two components to each other as explained above. Moreover, a glue which hardens is added to the gap 52 between the lens tube 28 and guide tube 22. For example, a UV cross-linking glue can be used for this so that fast cross-linking can be achieved, possibly using a UV laser, when a material translucent to the laser light is used for the guide tube 22. Preferably, the laser radiation is coupled into the region of the outer groove 48 so that the path traveled in the material of the guide tube 22 and the associated absorption of the laser light in the material is minimal. Moreover, the lens tube 28 can be connected to the guide tube 22 by welding if the two components are made of a metal. For this purpose, welding points are placed in a fillet between an outer lateral surface 32 of the lens tube 28 and an end face of the guide tube 22. A welding point 54 is for example depicted in FIG. 2. If a soldered connection is created, it is a soldering point. The welded or soldered connection can also be created with the assistance of a laser. Consequently, the lens tube 28 and the guide tube 22 can be fixed by, e.g., being welded, soldered and/or glued under the effect of laser radiation.

While there has been shown and described what is considered to be preferred embodiments, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### REFERENCE NUMBER LIST

2 Endoscope

4 Shaft

6 Housing

8 Eyepiece

10 Light source

12 Connecting cable

14 Camera head

16 Connection

20 Imaging unit

22 Guide tube

24 Image sensor

26 Optical element

28 Lens tube

30 Inner chamber

32 Outer lateral surface

34 Bar

36 Groove

38 Inner lateral surface

40 Side flank

40a First side flank

40b Second side flank

42 Side wall

42a First side wall

42b Second side wall

44 End face

46 Base

48 Outer groove

50 Outer lateral surface

52 Gap

54 Welding point

L1 Longitudinal direction of the lens tube

L2 Longitudinal direction of the guide tube

R Radial direction

R1 First parallel direction

R2 Second parallel direction

Z Center

α First angle

β Second angle

1. An imaging unit comprising:
a guide tube having an inner lateral surface extending in a longitudinal direction of the guide tube, the inner lateral surface defining an inner chamber;
a lens tube having an outer lateral surface extending in a longitudinal direction of the lens tube, the lens tube being at least sectionally accommodated within the inner chamber of the guide tube; and
at least one optical element which is accommodated in the lens tube;
wherein the lens tube comprises a plurality of bars disposed on the outer lateral surface, each of the plurality of bars interacting with a corresponding one of a plurality of grooves recessed in the inner lateral surface of the guide tube.

2. The imaging unit according to claim 1, wherein the plurality of bars and the plurality of grooves have complementary shapes in a cross-section lying perpendicular to one or more of the longitudinal direction of the lens tube and the longitudinal direction of the guide tube.

3. The imaging unit according to claim 2, wherein the complimentary shapes of one or more of the plurality of bars and the plurality of grooves are trapezoidal in a cross-section lying perpendicular to one or more of the longitudinal direction of the lens tube and the longitudinal direction of the guide tube, wherein one or more of at least one side flank of each of the plurality of bars and at least one side wall of each of the plurality of grooves is angled toward a center of a respective one of the plurality of bars and toward a center of a respective one of each of the plurality of grooves, such that in cross-section, one or more of the at least one side flank of the plurality of bars taper toward an end face and the at least one wall of the plurality of grooves narrow toward a base.

4. The imaging unit according to claim 3, wherein one or more of the at least one side flank of each of the plurality of bars and the at least one side wall of each of the plurality of grooves are angled at a predetermined inclination relative to a radial direction which runs radially starting from a center of one of the lens tube and the guide tube and intersects with one or more of a middle of the end face of a respective one of the plurality of bars and a middle of the base of a respective one of the plurality of grooves.

5. The imaging unit according to claim 4, wherein the predetermined inclination is in a range between 30° and 60°.

6. The imaging unit according to claim 5, wherein the predetermined inclination is in a range between 35° and 55°.

7. The imaging unit according to claim 6, wherein the predetermined inclination is in a range between 40° and 50°.

8. The imaging unit according to claim 7, wherein the predetermined inclination is at least approximately 45°.

9. The imaging unit according to claim 1, wherein the guide tube comprises an outer groove which extends in the longitudinal direction of the guide tube and is recessed in an outer lateral surface of the guide tube, wherein, the outer groove extends in a region of the guide tube such that a width of the outer groove at least partially overlaps a corresponding one of the plurality of grooves recessed in the inner lateral surface in the peripheral direction.

10. The imaging unit according to claim 9, wherein the width of the outer groove is at least a width of a corresponding one of the plurality of grooves recessed in the inner lateral surface in the peripheral direction.

11. The imaging unit according to claim 1, wherein the guide tube is formed of a translucent material, and the lens tube is formed of a material that is substantially light-absorbent.

12. An endoscope comprising the imaging unit according to claim 1.

13. A method for producing the imaging unit according to claim 1, the method comprising:
at least sectionally introducing the lens tube into the inner chamber surrounded by the guide tube such that each of the plurality of bars engages one of each of the plurality of grooves; and
fixing the optical element by connecting the lens tube to the guide tube.

14. A method for producing the imaging unit according to claim 3, the method comprising:
at least sectionally introducing the lens tube into the inner chamber surrounded by the guide tube such that each of the plurality of bars engages one of each of the plurality of grooves;
fixing the optical element by connecting the lens tube to the guide tube;
rotating the lens tube about the longitudinal direction of the lens tube relative to the guide tube such that the side flank of each of the plurality of bars comes into contact with a corresponding one of the angled side wall of the groove to center the optical element; and
displacing the lens tube relative to the guide tube in the longitudinal direction of the guide tube to set a desired longitudinal position of the lens tube.

15. The method according to claim 13, wherein the guide tube is made of a material translucent to the laser radiation and the lens tube is made of a material that substantially absorbs the laser radiation, wherein the fixing further comprises:
one of welding, soldering and gluing the lens tube and guide tube to each other under the effect of laser radiation, and
irradiating the guide tube with the laser radiation in the region of an outer groove to fix the lens tube to the guide tube.
